(19) United States
(12) Patent Application Publication
(10) Pub. No.: US 2008/0097203 A1
(43) Pub. Date: Apr. 24, 2008

(54) Standardized Digital Image Viewing with Ambient Light Control

(75) Inventors: Nadine Nereson, Snohomish, WA (US); David Rust, Seattle, WA (US); Tanar Ulric, Bothell, WA (US)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(73) Assignee: KONINKLIJKE PHILIPS ELECTRONICS N.V., EINDHOVEN (NL)

(21) Appl. No.: 11/571,689

(22) PCT Filed: Jun. 23, 2005
(86) PCT No.: PCT/IB05/52078

§ 371 Date: Jan. 5, 2007

Related U.S. Application Data
(60) Provisional application No. 60/587,847, filed on Jul. 13, 2004.

(51) Int. Cl. A61B 8/00

(52) U.S. Cl. 600/437

(57) ABSTRACT

An ultrasonic diagnostic imaging system is described which produces images in accordance with a display standard such as the DICOM standard. The DICOM standard images may be exported and reproduced on other display devices such as workstations and film or image printers. The standardized images produced by the system are transformed into unique driving levels which are characteristic of the system display device for viewing. The transform is user controllable for viewing standardized images under differing ambient light conditions.

This invention relates to medical diagnostic imaging systems and, in particular, to ultrasonic diagnostic imaging systems that enable the transfer and viewing of standardized images while allowing user control for variable ambient lighting conditions.

The acquisition, storage and viewing of digitized images is now a staple of medical diagnostic imaging. In ultrasound the use of digital images began over twenty years ago with the advent of digital scan converters. By digitizing the pixel values of an image, the image can be transferred, stored and reproduced with quantified accuracy. Standards have been put in place in many countries for the handling of digital diagnostic images. In the United States the Digital Imaging and Communications in Medicine (DICOM) standard has been developed and implemented, principally for standards pertinent to the transfer and storage of medical images. Important for the diagnoses made with DICOM standard images is the manner in which such images are presented for diagnosis. It is important for medical diagnostic images to be displayed with uniform visual consistency which leads to consistent diagnoses. An image displayed on an ultrasound monitor should have the same visual appearance when transferred and viewed on a diagnostic workstation or printed on film or photographic paper.

A part of the DICOM standard which deals with the visual presentation of images is PS 3.14. This part of the standard specifies a function that relates pixel values to displayed luminance levels. Specifically, PS 3.14 provides an objective, quantitative mechanism for mapping digital image values into a given range of luminance levels. By using a known functional relationship between pixel values and luminance levels, an image can be displayed and viewed on a different device or medium with the same diagnostic value it possesses on its original acquisition device.

One variable that PS 3.14 is designed to eliminate is the variability of user preferences which a user may employ to adjust an image to what the user personally feels is a more diagnostic presentation. One environmental variable which can motivate a user to make such adjustments is the lighting in the room or lab where the patient is being examined. In some instances the room may be brightly lighted to make the patient feel more comfortable and at ease, for example. In other instances the room may be more dimly lit, enabling subtle details in the displayed image to be more readily discerned by the diagnostician. In yet other instances the images may be acquired in a brightly lighted room, then transferred electronically to a workstation in a dimly lit diagnostic lab for reading by a diagnosing physician. In these variable conditions the sonographer will want to adjust the image display controls such as brightness and contrast to present an image which he or she feels is most diagnostic. The image must then be transferable to other devices or viewing media where it retains the same diagnostic value as it did to the original imaging system operator.

In accordance with the principles of the present invention, an ultrasonic diagnostic imaging system produces images for transfer and viewing on different media in accordance with a visual perception standard such as DICOM. A processor is provided for translating standardized images to the display function of the imaging system display device. A system user control or ambient light sensor is provided which enables the standardized images to be displayed on the imaging system display device with a display function that is modified to account for different ambient light conditions. The user can therefore view images on the imaging system which are diagnostic in a variety of ambient light conditions, and can export or print images with a standardized visual perception and diagnostic value.

In the drawings:

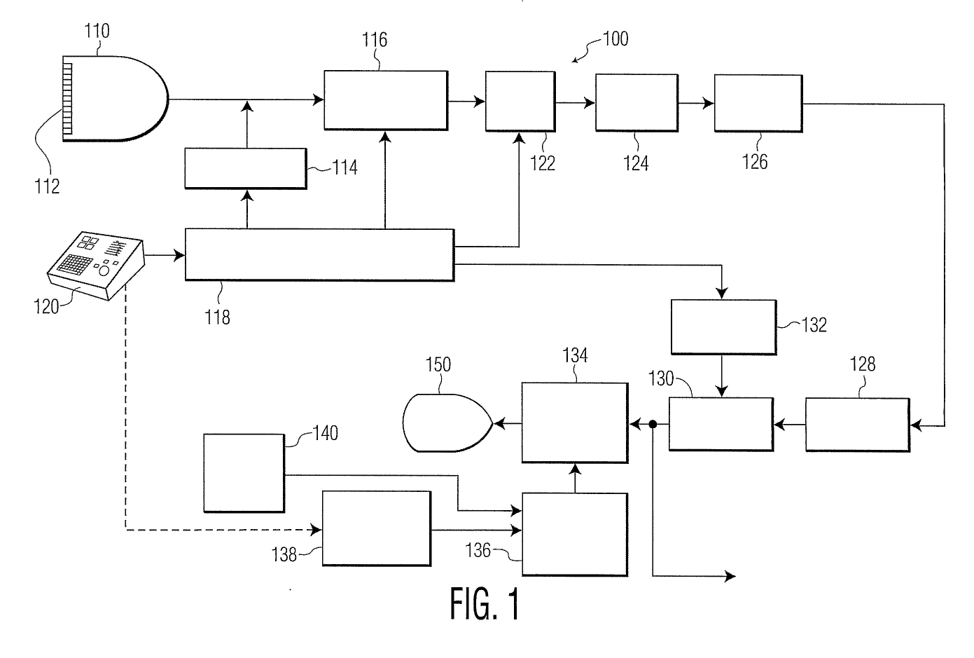

FIG. 1 illustrates in block diagram form an ultrasonic diagnostic imaging system constructed in accordance with the principles of the present invention.

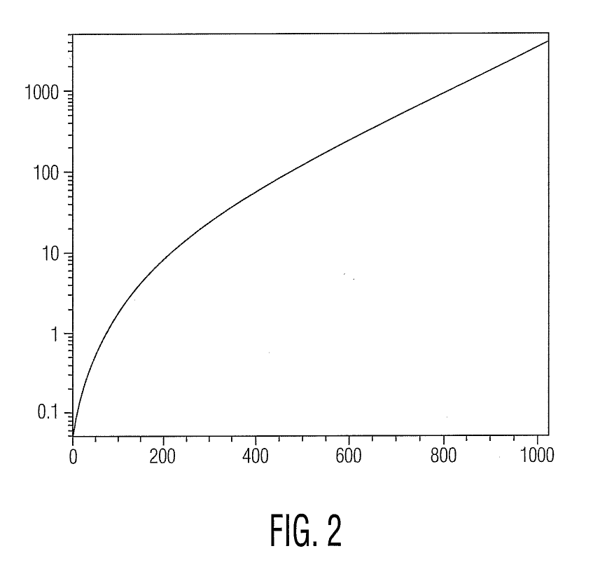

FIG. 2 graphically illustrates a standardized grayscale display function of luminance versus luminance differences that are just barely perceptible by a human observer.

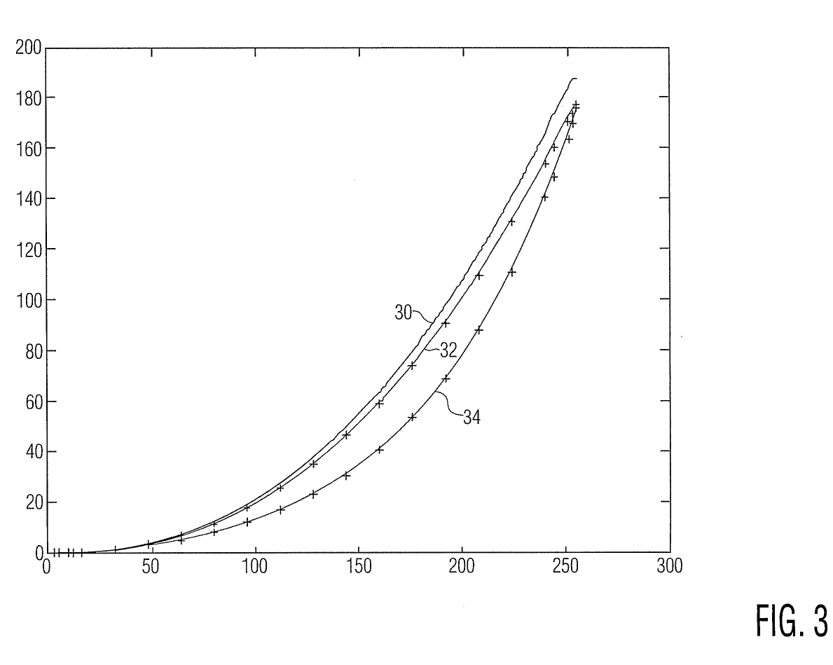

FIG. 3 graphically illustrates the translation of a standardized display function to the display function of an imaging system display device.

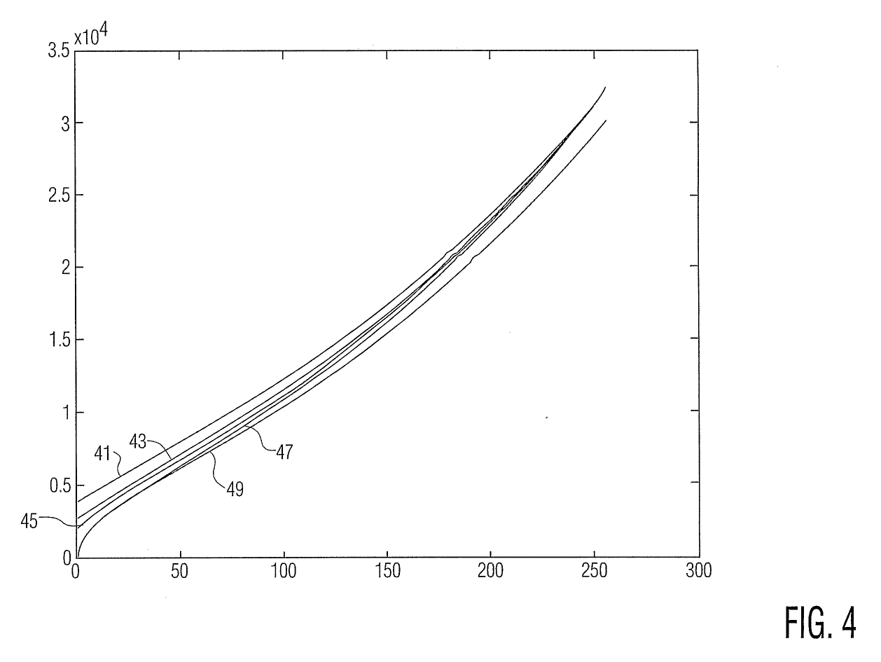

FIG. 4 graphically illustrates a series of display functions for different ambient light conditions which can be selected by a user for control of a display device.

Referring first to FIG. 1, an ultrasonic diagnostic imaging system 100 constructed in accordance with the principles of the present invention is shown in block diagram form. The imaging system 100 includes a scanhead 110 having an array transducer 112 that transmits beams at different angles over an image field. The transmission of the beams is controlled by a transmitter 114, which controls the frequency, phasing and time of actuation of each of the elements of the array transducer 112 so each beam is transmitted from a predetermined origin along the array and at a predetermined angle. The echoes returned from along each beam direction are received by the elements of the array, digitized by analog-to-digital conversion, and coupled to a digital beamformer 116. The digital beamformer 116 delays and sums the echoes from the array elements of the transducer 112 to form a sequence of focused, coherent digital echo samples along each scanline or beam direction. The sequence of samples are used to form respective image frames corresponding to the beams formed by the beamformer 116. The transmitter 114 and beamformer 116 are operated under control of a system controller 118, which in turn is responsive to the settings of controls on a user interface 120 operated by the user of the ultrasound system 100. The system controller 118 controls the transmitter 114 to transmit the desired number of scanline groups at the desired angles, transmit energies and frequencies. The system controller 118 also controls the digital beamformer 116 to properly delay and combine the received echo signals for the apertures and image depths used.

The scanline echo signals are filtered by a programmable digital filter 122, which defines the band of frequencies of interest. When imaging harmonic contrast agents or performing tissue harmonic imaging, the passband of the filter 122 is set to pass harmonics of the transmit band. The filtered signals are then detected by a detector 124. For B mode imaging, the detector 124 performs amplitude detection of the echo signal envelope. For Doppler imaging, ensembles of echoes are assembled for each point in the image and are Doppler processed to estimate the Doppler shift or Doppler power intensity. The echo data from the scanlines of an image are collected in an image memory 126. The data of an image is coupled to a scan converter 128 where the echo data is arranged-in the desired image format such as a rectangular linearly scanned image or a sector-shaped image.

The echo signals are converted to a range of display values in a process known as mapping. A set of grayscale image values undergo a grayscale mapping process 130 and Doppler values generally undergo a color mapping process. Grayscale mapping usually includes a logarithmic conversion of the echo values to translate the echo values to a range of values which are more readily discerned by the human eye. Grayscale mapping with logarithmic conversion will map lower luminance levels to a range of values in which slightly different darker values can be more easily distinguished, enabling better definition of more subtle tissue features. In accordance with the present invention the echo values are mapped to a standardized grayscale display function such that individual steps in the grayscale range produce equally spaced differences in visually perceived grayscale levels to the average human observer In a constructed embodiment of the present invention a grayscale image is mapped to the standard display function (SDF) of luminance display values of the DICOM standard. The luminance values of the SDF are those defined in PS 3.14. FIG. 2 illustrates a curve of logarithmically scaled luminance values versus an index of just-noticeable differential values of the DICOM standardized display function.

The image mapped to the SDF can then be transferred to external networks, storage devices and display devices such as workstations, paper printers, and film printers. When these devices are configured to respond to DICOM standard images, the images can be reproduced to same diagnostic value. The images may be shown on emissive displays such as workstation monitor or LCD display in a darkened room or printed on transmissive film and viewed on a radiology light-box or printed on glossy or non-glossy photographic paper with the same diagnostic presentation in each case. This is done by applying the standard DICOM images to the characteristic display curve of the respective display device, which translates the standard image to the known display characteristic of the display device. The images will exhibit the same diagnostic value, within the limitations of the display device, for a variety of display devices on which they are displayed.

In accordance with a further aspect of the present invention the user has the ability to select a map which the user feels best presents the diagnostic aspects of the images. This is done by selecting a new mapping function from a grayscale maps store 132 through the user control panel 120 and the system controller 118. Such user selectable maps are generally empirically derived from observations of how users desire their images to appear in specific applications. In vascular applications for instance a user will generally want low levels suppressed and vessel walls enhanced and sharply defined in white. In breast and liver images for instance a user will generally want low grayscale levels distinctly distributed so as to better discern subtle contrast differences in low level regions of the image. When a new map is selected by the user, the new mapping function replaces the previous mapping function used which in the first instance is the default map for the clinical application being performed. The range of luminance values of the new map is shown on the luminance bar displayed adjacent to the image and the identification of the map used may be stored along with the image for subsequent use. The stored mapping function, like the default map of the grayscale mapping function 130, is generally a lookup table whereby an input echo value will address an output luminance value of the grayscale map.

In accordance with a further aspect of the present invention the image which has been mapped to the standardized display function (SDF) is applied to a SDF/DD transform processor 134 which transforms an image mapped to standardized luminance values to a range of display values suitable for the display device 150 of the ultrasound system 100. For example, the image data applied at the input of the transform processor 134 may be mapped to a series of discrete luminance values which graphically plot to a standard curve 30 of luminance values for a typical CRT display device as shown in FIG. 3. A different display device 150 however may respond to a series of digital driving levels (DDLs) which plot to luminance values in accordance with a display function that is unique to the different display device, as illustrated by the flat panel display device response curve 32. In order to faithfully reproduce the luminance levels of the standardized image on a unique display device 150 the values of the SDF curve must be translated from those of the device-specific response curve 32 to those of a curve 34, which represent the luminance range of an ultrasound image in a linear scale. This is preferably done by a lookup table of output DDL values which are addressed by input luminance values of the standardized image at the input of the transform processor 134. Another display device may have a different display response and a translation will then be performed from the SDF curve to the values of another device function in order to accurately drive the different display device. When the DDL values produced by the transform processor 134 are applied to the display device 150, the display is driven by drive levels specific to the device which cause the display to produce images with luminance levels conforming to the human perception levels of the DICOM display standard.

In accordance with another aspect of the present invention the ultrasound system user can change the display function used for the display device 150 in response to ambient light levels. This allows the user to adjust the brightness of the display of a standardized image in consideration of the light level in the room where the ultrasound system is used. As the lighting in a room becomes brighter the lower dynamic range of the image display deteriorates, principally due to the reflection of room light by the display surface. This will cause darker values which are close enough to satisfy the just-noticeable differential display criterion to become visually indistinct, thereby reducing the diagnostic value of the image in areas where subtle tissue differences are present. This problem is more severe in the case of systems with CRT monitors, as the glass of the monitor will reflect an appreciable amount of light as compared with flat panel displays such as LCD displays, where filters and lenses will absorb more of the ambient light.

The conventional way of addressing room lighting differences is to provide brightness and contrast controls on the display device which the user can adjust in accordance with ambient light levels. As the room becomes brighter the user can adjust the brightness and contrast controls of the display. This approach however is unlikely to adjust the image luminance in the manner which is needed, which is to restore just-noticeable differences to the lower luminance levels in particular. To accomplish this desired result the present inventors have empirically measured the light returned from the display device 150 with a photometer under different ambient light conditions. These conditions varied over five ambient light levels, from a very dimly lit room to a very brightly lighted room. The light levels of different grayscale values were recorded and used to empirically create five different curves in lookup table form as shown in FIG. 4. The curves shown in this drawing are a function of p-values, which are device-independent standardized values, versus digital driving levels for the particular display device 150. These curves will boost low level response, the most sensitive to light level changes, as ambient light levels increase. The curve 41 for instance is relatively linear throughout its range. This curve would be used in a brightly lit room where degradation of the display dynamic range at low luminance levels requires more compensation. The higher numbered curves are used for progressively dimmer ambient room lighting levels. The curve 49 for instance applies a more rapid change between consecutive low grayscale levels, as is evident from the steeply curved shape near the origin of the graph. This display function will impose the greater differentiation in low level driving values needed to maintain the diagnostic value of the displayed image, particularly the low luminance levels, in a dimly lighted room.

As the ambient light level in a room is increased or decreased, the user will adjust the displayed image by manipulation of a user brightness control 138 on the control panel 120 or user interface, thereby selecting a new ambient light function from a selection of ambient light functions 136. The new ambient light function (as indicated by the group of curves 41-49) is then used to convert the standardized image display function SDF into an ambient light-adjusted display function for the display device 150. It will be appreciated that an embodiment of the invention may use a single baseline SDF/DD transform function in the transform processor 134 which is augmented by one of the ambient light functions of the ambient light function store 136. Alternatively, each lookup table of the ambient light function store 136 may effect the total transform from the standard function to the driving levels needed for a particular ambient light condition, in which case the single lookup table selected by the user performs the full transform for the display. Such implementation choices are a matter of design and system architecture considerations.

It will further be appreciated that the ultrasound system may be equipped with an ambient light sensor 140, enabling the system to automatically select and apply the appropriate ambient light transform function 136 based upon the sensed ambient lighting conditions. Preferably this automatic mode of adjustment may be turned on, or turned off if the user prefers to adjust the display manually.

What is claimed is:

1. An ultrasonic diagnostic imaging system which produces images with a visual appearance defined by a display standard comprising:
an ultrasound probe which receives echo signals from a subject;
a processor coupled to the probe which is responsive to received echo signals and produces image values;
a mapping processor responsive to the image values which maps the image values using a desired mapping function which satisfies a display standard;
a communication port responsive to the mapping processor which provides images which satisfy the display standard to external storage or display devices;
a display device for the imaging system; and
a transform processor responsive to the mapping processor and coupled to the imaging system display device which transforms an image which satisfied the display standard to a characteristic display function of the display device.

2. The ultrasonic diagnostic imaging system of claim 1, further comprising a plurality of ambient light functions responsive to a user control and coupled to the transform processor which enables the transform of a standardized image to a display function for the display device for different ambient light conditions.

3. The ultrasonic diagnostic imaging system of claim 1, wherein the transform processor further comprises a lookup table responsive to an image which satisfies a standard display function for the production of driving level signals for the imaging system display device.

4. The ultrasonic diagnostic imaging system of claim 3, wherein the imaging system display device comprises a flat panel display.

5. The ultrasonic diagnostic imaging system of claim 2, wherein the plurality of ambient light functions are stored as lookup tables.

6. The ultrasonic diagnostic imaging system of claim 5, wherein the ambient light functions augment a function which transforms a standardized image into driving levels for a particular display device.

7. The ultrasonic diagnostic imaging system of claim 5, wherein the ambient light functions each perform a transform of a standardized image into driving levels for a particular display device for a different ambient light condition.

8. The ultrasonic diagnostic imaging system of claim 1, wherein the mapping processor is responsive to image values for mapping image values to a grayscale map.

9. The ultrasonic diagnostic imaging system of claim 8, further comprising a source of different grayscale maps coupled to the mapping processor; and
a user control, coupled to the source of different grayscale maps, for selecting a particular grayscale map for use by the mapping processor.

10. The ultrasonic diagnostic imaging system of claim 1, wherein the mapping processor further comprises a logarithmic converter which acts to convert image values to a logarithmic range of values.

11. The ultrasonic diagnostic imaging system of claim 1, wherein the mapping processor further comprises a processor which is responsive to image values to map the image values to a mapping function of just-noticeable differential display values.

12. The ultrasonic diagnostic imaging system of claim 1, wherein the transform processor further comprises a processor which transforms a standardized image of just-noticeable differential display values to driving levels for a display which are characteristic of the display and reproduce an image of just-noticeable differential luminance display levels.

13. The ultrasonic diagnostic imaging system of claim 1, wherein the communication port is coupled to a network which includes at least one of an emissive image display and a printed image display.

14. The ultrasonic diagnostic imaging system of claim 1, further comprising:
an ambient light sensor; and
a plurality of ambient light functions responsive to the ambient light sensor and coupled to the transform processor which enables the transform of a standardized image to a display function for the display device for different ambient light conditions.
